(19) United States
(12) Patent Application Publication
(10) Pub. No.: US 2022/0404379 A1
(43) Pub. Date: Dec. 22, 2022

(54) SEQUENCING METHOD, SYSTEM AND KIT OF LOW MOLECULAR WEIGHT HEPARIN OLIGOSACCHARIDES

(71) Applicants: Shandong University, Shandong (CN); National Institute of Metrology, China, Beijing (CN)

(72) Inventors: Lianli Chi, Shandong (CN); Deling Shi, Shandong (CN); Bing Zhang, Shandong (CN); Tianji Zhang, Beijing (CN); Fei Li, Shandong (CN); Qingqing Chen, Shandong (CN)

(73) Assignees: Shandong University, Shandong (CN); National Institute of Metrology, China, Beijing (CN)

(21) Appl. No.: 17/577,348

(22) Filed: Jan. 17, 2022

(30) Foreign Application Priority Data
Jun. 22, 2021 (CN) 202110691317.4

(51) Int. Cl. G01N 33/94; G16C 20/20; G16C 20/90; G06F 16/11

(52) U.S. Cl. CPC G01N 33/94; G16C 20/20; G16C 20/90; G06F 16/122

(57) ABSTRACT

A sequencing method, system and kit of low molecular weight heparin (LMWH) oligosaccharides are provided. The sequencing method includes: a sample preparation step: isolating or preparing a group of LMWH oligosaccharide mixture samples; a sample treatment step: performing complete enzymatic digestion and nitrous acid degradation on the LMWH oligosaccharide mixture samples to obtain an enzymatically digested eight-common-heparin-disaccharide array, a 3-O-sulfate group array, a 1,6-anhydro structure array, a nitrous acid degradation array, respectively; a data processing step: obtaining a disaccharide isomeric unit array according to the enzymatically digested eight-common-heparin-disaccharide array and the nitrous acid degradation array; a sequence database building step: building a sequence database according to the degree of polymerization of the oligosaccharide mixture, the disaccharide isomeric unit array, the 3-O-sulfate group array, and the 1,6-anhydro structure array; and a specific result output step: screening the sequence database according to input qualification information and then outputting a specific result file.

### CROSS-REFERENCE TO RELATED APPLICATION

This application claims the priority benefit of China application serial no. 202110691317.4, filed on Jun. 22, 2021. The entirety of the above-mentioned patent application is hereby incorporated by reference herein and made a part of this specification.

### BACKGROUND

### Technical Field

The present disclosure relates to the sequencing of low molecular weight heparin oligosaccharides, and specifically and particularly relates to a computer-aided sequencing method and system of low molecular weight heparin oligosaccharides, and a sequencing kit for sequencing oligosaccharides of low molecular weight heparin drugs.

### Description of Related Art

In the field of pharmaceutical chemistry, carbohydrate drugs are paid more and more attention, and their structural characterization is very important. However, due to the structural complexity and heterogeneity of natural polysaccharides, there is a lack of effective sequence analysis method, which severely limits the research on the structure-activity relationship and the quality control of drugs. Carbohydrate sequencing has always been a major challenge in the research of carbohydrate drugs, especially in the research of low molecular weight heparin (LMWH) drugs.

Heparin is a linear polysaccharide which is composed of L-iduronic acid or D-glucuronic acid (1→4) D-glucosamine disaccharide units, has polydispersity, and has a biological function of promoting the anticoagulation activity due to its specific sequence bound to antithrombin III. LMWHs are a class of anticoagulant drugs with low molecular weights that are obtained from heparin under different degradation conditions, and include Enoxaparin sodium, Nadroparin calcium, Dalteparin sodium, and other varieties according to technologies and structures. Compared with heparin, LMWHs have advantages of high bioavailability, high antithrombotic effect, long plasma half-life, and low bleeding risk, and are a class of anticoagulant drugs widely used in clinical practice. A uronic acid residue of a disaccharide repeating unit of a heparin may be substituted with a 2-O-sulfate group, and a glucosamine residue may be substituted with N-acetyl, an N-sulfate group, a 6-O-sulfate group or 3-O-sulfate group. The degradation process of heparin will also change terminals of oligosaccharides. Therefore, LMWH is a highly isomeric mixture with a complex molecular structure, and its oligosaccharide sequencing is a key and difficult point in the research of LMWH drugs.

The existing methods for characterizing oligosaccharides of LMWH drugs mainly adopts mass spectrometry and nuclear magnetic technologies to map a monosaccharide composition, basic building blocks, partially enzymatically digested oligosaccharide fragments, an intact chain, etc. of the LMWH drugs. These analysis methods are mainly focused on the overall analysis of the mixture, and cannot give a complete sequence analysis of the oligosaccharides.

The sequencing methods of heparin and LMWH oligosaccharides mainly include the steps of obtaining single oligosaccharides from naturally mixed oligosaccharides by a complex isolation method, or obtaining pure products by a synthesis method, and then performing structural analysis by a method such as mass spectrometry (MS) or nuclear magnetism (NMR).

Commonly used isolation and purification methods of oligosaccharides include: size exclusion chromatography (SEC), affinity chromatography, strong anion exchange (SAX), reversed-phase ion pair (RPIP), hydrophilic interaction chromatography (HILIC), capillary electrophoresis (CE), etc. Complex mixed heparin oligosaccharides are isolated by one or more of the above isolation technologies to obtain relatively pure oligosaccharides, and then the oligosaccharides are sequenced by a plurality of analysis methods such as mass spectrometry and nuclear magnetism. Due to the structural complexity and heterogeneity of LMWH oligosaccharides, isolation of the oligosaccharides requires complex procedures, and costs a lot of time and effort of researchers. But even so, it is still difficult to obtain enough pure products for oligosaccharide sequencing. At present, research on the synthesis of heparin oligosaccharides has made slow progress, and it is still difficult to realize the chemosynthesis or biosynthesis of heparin oligosaccharides.

In view of the above, it is difficult to characterize the structure of LMWH oligosaccharides by the current techniques, and especially with the extension of the oligosaccharides, the number of its isomers increases exponentially, making the characterization of a group of oligosaccharide mixtures with similar structures and compositions becomes extremely difficult. At present, there is no technology that can easily and quickly obtain the sequence information of a group of mixed LMWH oligosaccharides with similar compositions and structures, there is an urgent need for a simple, fast, time-saving, and labor-saving method to interpret the structural information represented by these complex molecules.

### SUMMARY

Aiming at the above problems, the present disclosure provides a sequencing method of low molecular weight heparin (LMWH) oligosaccharides, including:

a sample preparation step: isolating or preparing a group of LMWH oligosaccharide mixture samples according to experimental requirements;

a sample treatment step: performing complete enzymatic digestion and nitrous acid degradation on the LMWH oligosaccharide mixture samples to obtain an enzymatically digested 8-common-heparin-disaccharide array, a 3-O-sulfate group array, a 1,6-anhydro structure array, a nitrous acid degradation array, respectively;

a data processing step: calculating IdoA/GlcA of different disaccharides according to the eight-common-heparin-disaccharide array and the nitrous acid degradation array to obtain a disaccharide isomeric unit array;

a sequence database building step: building a sequence database according to the degree of polymerization of the oligosaccharide mixture, the disaccharide isomeric unit array, the 3-O-sulfate group array, and the 1,6-anhydro structure array; and

a specific result output step: screening the sequence database according to input qualification information and then outputting a specific result file.

In the above sequencing method of LMWH oligosaccharides, the sample treatment step includes:

an enzymatic digestion treatment step: performing complete enzymatic digestion on the LMWH oligosaccharide mixture samples with a mixture of heparinases I, II, and III to obtain the enzymatically digested eight-common-heparin-disaccharide array, the 3-O-sulfate group array, and the 1,6-anhydro structure array; and

a nitrous acid degradation treatment step: degrading the LMWH oligosaccharide mixture samples with nitrous acid to obtain the nitrous acid degradation array.

In the above sequencing method of LMWH oligosaccharides, the sequence database building step includes:

a 1,6-anhydro structure-excluding theoretical sequence database building step: building a 1,6-anhydro structure-excluding theoretical sequence database according to the 3-O-sulfate group array and the disaccharide isomeric unit array;

a 1,6-anhydro structure-including theoretical sequence database building step: building a 1,6-anhydro structure-including theoretical sequence database according to the 3-O-sulfate group array, the 1,6-anhydro structure array, and the disaccharide isomeric unit array; and

a combination step: correlating and combining the 1,6-anhydro structure-excluding theoretical sequence database and the 1,6-anhydro structure-including theoretical sequence database to obtain the sequence database.

In the above sequencing method of LMWH oligosaccharides, the combination step includes combining results of the 1,6-anhydro structure-excluding theoretical sequence database and results of the 1,6-anhydro structure-including theoretical sequence database, and sorting the results according to the value of each sequence to obtain the sequence database.

In the sequencing method of LMWH oligosaccharides, the result file includes: 1,6-anhydro structure-including sequences—generation time, 1,6-anhydro structure-excluding sequences—generation time, total sequences—sorted in decreasing order based on the content—generation time, selected sequences—at least one of generation time and a log file that includes at least one of the total number of calculated sequences, the type and value of the disaccharide isomeric unit array, the total record number of total sequences, the total record number of 1,6-anhydro structure-excluding sequences, and the total record number of 1,6-anhydro structure-including sequences.

In the above sequencing method of LMWH oligosaccharides, the 1,6-anhydro structure-excluding theoretical sequence database building step includes: if all elements in the 3-O-sulfate group array are 0, setting the number of selected elements as dp/2, selecting dp/2 elements from the disaccharide isomeric unit array, arranging all the selected elements in a row in order (the same element can be selected repeatedly), and calculating and outputting string concatenation of various elements and a product of values of various elements; and if all elements in the 3-O-sulfate group array are not 0, setting the number of selected elements as (dp−4)/2, selecting at least one element from the 3-O-sulfate group array, selecting (dp−4)/2 elements from the disaccharide isomeric unit array, arranging the remaining selected elements in a row in order, and calculating and outputting string concatenation of various elements and a product of values of various elements, wherein dp is the degree of polymerization of the oligosaccharide mixture.

In the above sequencing method of LMWH oligosaccharides, the 1,6-anhydro structure-including theoretical sequence database building step includes:

if an IdoA2S-GlcNS6S-IdoA2S-ManNS-1,6-anhydro group in the 1,6-anhydro structure array is greater than 0 and all elements in the 3-O-sulfate group array are 0, setting the number of selected elements as (dp−4)/2, selecting one element (IdoA2S-GlcNS6S-IdoA2S-ManNS-1,6-anhydro) from the 1,6-anhydro structure array, selecting (dp−4)/2 elements from the disaccharide isomeric unit array, setting the element (IdoA2S-GlcNS6S-IdoA2S-ManNS-1,6-anhydro) as the rightmost element of a string, arranging the (dp−4)/2 elements that are selected from the disaccharide isomeric unit array in a row in order on the left side of the element, and calculating and outputting string concatenation of various elements and a product of values of various elements;

if an IdoA2S-GlcNS6S-IdoA2S-ManNS-1,6-anhydro group of the 1,6-anhydro structure array is greater than 0 and all elements in the 3-O-sulfate group array are not 0, setting the number of selected element as (dp−4−4)/2, selecting one element from the 3-O-sulfate group array, selecting one element (IdoA2S-GlcNS6S-IdoA2S-ManNS-1,6-anhydro) from the 1,6-anhydro structure array, selecting (dp−4−4)/2 elements from the disaccharide isomeric unit array, setting IdoA2S-GlcNS6S-IdoA2S-ManNS-1,6-anhydro as the rightmost element of a string, arranging the remaining selected elements in a row in order, and calculating and outputting string concatenation of various elements and a product of values of various elements;

if a GlcA-GlcNS/ManNS-1,6-anhydro group or an IdoA2S-GlcNS-1,6-anhydro group in the 1,6-anhydro structure array is greater than 0 and all elements in the 3-O-sulfate group array are 0, setting the number of selected elements as (dp−2)/2, selecting one element (GlcA-GlcNS/ManNS-1,6-anhydro or IdoA2S-GlcNS-1,6-anhydro) from the 1,6-anhydro structure array, selecting (dp−2)/2 elements from the disaccharide isomeric unit array, setting GlcA-GlcNS/ManNS-1,6-anhydro or IdoA2S-GlcNS-1,6-anhydro as the rightmost element of a string, arranging the (dp−2)/2 elements that are selected from the disaccharide isomeric unit array in a row in order on the left side of the element, and calculating and outputting string concatenation of various elements and a product of values of various elements; and

if a GlcA-GlcNS/ManNS-1,6-anhydro group or an IdoA2S-GlcNS-1,6-anhydro group in the 1,6-anhydro structure array is greater than 0 and all elements in the 3-O-sulfate group array are not 0, setting the number of selected elements as (dp−4−2)/2, selecting one element (IdoA-GlcNAc6S-GlcA-GlcNS3S6S or IdoA-GlcNS6S-GlcA-GlcNS3S6S or IdoA-GlcNAc6S-GlcA-GlcNS3S or IdoA2S-GlcNAc6S-GlcA-GlcNS3S6S or IdoA2S-GlcNS6S-GlcA-GlcNS3S) from the 3-O-sulfate group array, selecting one element (GlcA-GlcNS/ManNS-1,6-anhydro or IdoA2S-GlcNS-1,6-anhydro) from the 1,6-anhydro structure array, selecting (dp−4−2)/2 elements from the disaccharide isomeric unit array, setting GlcA-GlcNS/ManNS-1,6-anhydro or IdoA2S-GlcNS-1,6-anhydro as the rightmost element of a string, arranging the remaining selected elements in a row in order, and calculating and outputting string concatenation of various elements and a product of values of various elements.

The present disclosure also provides a sequencing system of LMWH oligosaccharides, including:

a sample preparation unit, configured to isolate or prepare a group of LMWH oligosaccharide mixture samples according to experimental requirements;

a sample treatment unit, configured to perform complete enzymatic digestion and nitrous acid degradation on the LMWH oligosaccharide mixture samples to obtain an enzymatically digested eight-common-heparin-disaccharide array, a 3-O-sulfate group array, a 1,6-anhydro structure array, and a nitrous acid degradation array, respectively;

a data processing unit, configured to calculate IdoA/GlcA of different disaccharides according to the eight-common-heparin-disaccharide array and the nitrous acid degradation array to obtain a disaccharide isomeric unit array;

a sequence database building unit, configured to build a sequence database according the degree of polymerization of the oligosaccharide mixture, the disaccharide isomeric unit array, the 3-O-sulfate group array, and the 1,6-anhydro structure array; and

a specific result output unit, configured to screen the sequence database according to input qualification information and then output a specific result file.

The present disclosure also provides a sequencing kit for sequencing oligosaccharides of LWMH drugs, including H₂SO₄, Ba(NO₂)₂, NaNO₂, Na₂CO₃, acetic acid, ammonium hydroxide, NaBH₄, heparinase I, heparinase II, heparinase III, and an enzymatic digestion buffer.

The above sequencing kit specifically includes:

1 mL of H₂SO₄ with a concentration of 0.5 moL; 1 mL of Ba(NO₂)₂ with a concentration of 0.5 moL; 1 mL of NaNO₂ with a concentration of 5.5 moL; 1 mL of Na₂CO₃ with a concentration of 1.0 moL; 1 mL of acetic acid with a concentration of 0.1 moL; 1 mL of ammonium hydroxide with a concentration of 0.1 moL; 1 g of NaBH₄; 20 mIU 50 μL*10 of heparinase I; 20 mIU 50 μL*10 of heparinase II; 20 mIU 50 μL*10 of heparinase III; and 1 mL of enzymatic digestion buffer.

The above sequencing kit is used to perform nitrous acid degradation on a LMWH drug to obtain a nitrous acid degradation product, and is used to perform complete enzymatic digestion on the LMWH drug to obtain a complete enzymatic digestion product.

The sequencing kit also includes two liquid chromatography columns of which one liquid chromatography column is Hypercarb Column (5 μm, 150 mm×4.6 mm) for analyzing the nitrous acid degradation product, and the other liquid chromatography column is Phenomenex Luna 3 μm HILIC 200 Å for analyzing the complete enzymatic digestion product.

Aiming at the deficiencies of the existing sequencing technologies of LMWH oligosaccharides, the present disclosure is directed to provide a simple, efficient, and high-throughput sequencing method, system and kit of LMWH drugs. The present disclosure is especially applicable to the sequencing of complex heparin oligosaccharide mixtures. According to the present disclosure, the sequencing kit is used to treat samples, and data is mainly processed and analyzed by computer software, that is, the sequencing kit and a computer software-aided data processing technology are combined, the high-throughput sequencing of mixed heparin oligosaccharides can be realized without isolation and purification of the oligosaccharides, thereby greatly reducing the workload of an analyst, reducing costs, improving working efficiency, and providing scientific researchers and R&D staffs of enterprises with a powerful sequencing product and technical supports.

It should be noted that the present disclosure emphasizes sequences rather than other physicochemical properties, such as the length, the molecular weight, the degree of sulfation, of oligosaccharides, and thus, the present disclosure can rapidly obtain all theoretical sequences and contents of a group of LMWH oligosaccharide mixtures and can realize rapid sequencing of a group of heparin oligosaccharide mixtures without purification of the heparin oligosaccharides.

Other features and advantages of the present disclosure will be described in the following description, and partially become obvious from the description, or understood by implementing the present disclosure. Objectives and other advantages of the present disclosure can be realized and obtained through structures indicated in the description, claims and drawings.

### BRIEF DESCRIPTION OF THE DRAWINGS

In order to explain technical solutions in the embodiments of the present disclosure or the prior art more clearly, the drawings that need to be used in the description of the embodiments or the prior art will be briefly described below. Obviously, the drawings in the following description are some of the embodiments of the present disclosure, and those of ordinary skill in the art can obtain other drawings based on these drawings without creative effort.

FIG. 1 is a flowchart of a sequencing method of low molecular weight heparin (LMWH) oligosaccharides according to the present disclosure;

FIG. 2 is a flowchart of sub-steps of S2 of FIG. 1;

FIG. 3 is a flowchart of sub-steps of S3 of FIG. 1;

FIG. 4 is a flowchart of result file output;

FIG. 5 is a schematic effect diagram of a result file;

FIG. 6 is an application process diagram of the present disclosure;

FIG. 7 is a schematic diagram of sequence database building; and

FIG. 8 is a schematic structural diagram of a sequencing system of LMWH oligosaccharides according to the present disclosure.

### DESCRIPTION OF THE EMBODIMENTS

In order to make the objectives, technical solutions, and advantages of the embodiments of the present disclosure clearer, the technical solutions in the embodiments of the present disclosure will be described clearly and completely with reference to the accompanying drawings in the embodiments of the present disclosure. Obviously, the described embodiments are some but not all of the embodiments of the present disclosure. Based on the embodiments of the present disclosure, all other embodiments obtained by those of ordinary skill in the art without creative effort shall fall within the scope of protection of the present disclosure.

The illustrative embodiments of the present disclosure and their descriptions are used to explain the present disclosure, but not intended to limit the present disclosure. In addition, elements/components with the same or similar reference numerals used in the drawings and the embodiments are used to represent the same or similar parts.

As used herein, “first”, “second”, “S1”, “S2”, etc. do not specifically refer to the order or the sequence, are not used to limit the present disclosure, but are merely used to distinguish elements or operations described in the same technical terms.

As used herein, the directional terms such as: up, down, left, right, front, and rear only indicate directions with reference to the drawings. Therefore, the used directional terms are used to describe but not to limit the present disclosure.

As used herein, “comprise”, “include”, “have”, “contain”, etc. are all open terms, which mean including but not limited to.

As used herein, “and/or” includes any one or a combination of all the described things.

As used herein, “a plurality of” includes “two” and “more than two”; and “a plurality of groups” includes “two groups” and “more than two groups”.

As used herein, the terms ““approximately”, “about”, etc. are used to modify any quantity that can be slightly changed or error, but these slight changes or errors will not change its essence. Generally, the range of the slight changes or errors modified by such terms may be 20% in some embodiments, 10% in some embodiments, 5% in some embodiments, or other values. Those skilled in the art should understand that the aforementioned values can be adjusted according to actual needs and are not limited thereto.

Some terms used to describe the present application will be discussed below or elsewhere in this description to provide those skilled in the art with additional guidance on the description of the present application.

First, the present disclosure discloses a sequencing kit, including: 0.5 moL H₂SO₄, 0.5 moL Ba(NO₂)₂, 5.5 moL NaNO₂, 1 moL Na₂CO₃, 0.1 moL acetic acid, 0.1 moL ammonium hydroxide, NaBH₄, heparinase I, heparinase II, heparinase III, an enzymatic digestion buffer, Superdex 30 column (16/60 m), and Phenomenex Luna 3 μm HILIC 200 Å (150×2.0 mm). The specific composition of various parts is shown in the table below.

Composition of Kit

| Name | Dosing | Note |
|---|---|---|
| 0.5 moL HS₂O₄ | 1 mL | Ready-to-use, mix |
| 0.5 moL Ba(NO₂)₂ | 1 mL | and use according to |
| 5.5 moL NaNO₂ | 1 mL | different proportions |
| 1 moL Na₂CO₃ | 1 mL | in the instruction, and |
| | | store at 4° C. |
| 0.1 moL acetic acid | 1 mL | None |
| 0.1 moL ammonium | 1 mL | None |
| hydroxide | | |
| NaBH₄ | 1 g | Ready-to-use, and |
| | | store at 4° C. |
| Heparinase I | 20 mIU (50 μL) * 10 | Ready-to-use, mix |
| Heparinase II | 20 mIU (50 μL) * 10 | according to |
| Heparinase III | 20 mIU (50 μL) * 10 | proportions, and store |
| Enzymolysis buffer | 1 mL | at 4° C. |
| Superdex 30 column (16/60 | 1 | Can be self-provided |
| m) | | |
| Phenomenex Luna 3 μm | 1 | Can be self-provided |
| HILIC 200 Å (150 × 2.0 mm) | | |

Users can select chromatographic columns as required.

It should be noted that the sequencing kit is used to sequence mixed oligosaccharides of low molecular weight heparin (LMWH) drugs, has a shelf life of 12 months, and shall be stored at −20° C. away from light. When the kit is transported over long distances, dry ices are required as a refrigerant. In the test, the kit is taken out of the refrigerator at −20° C. in advance, placed at 4° C., and mixed well by gentle shaking; an ice box is prepared, and experimental operations are performed on the ice box.

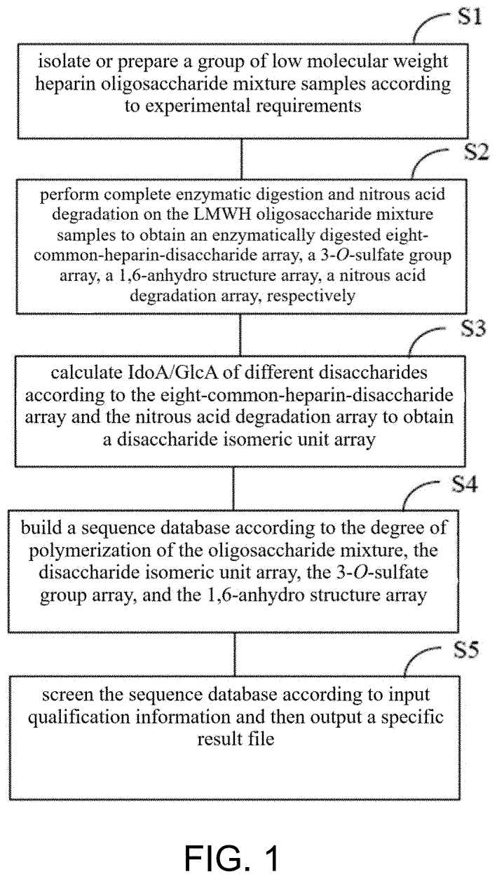

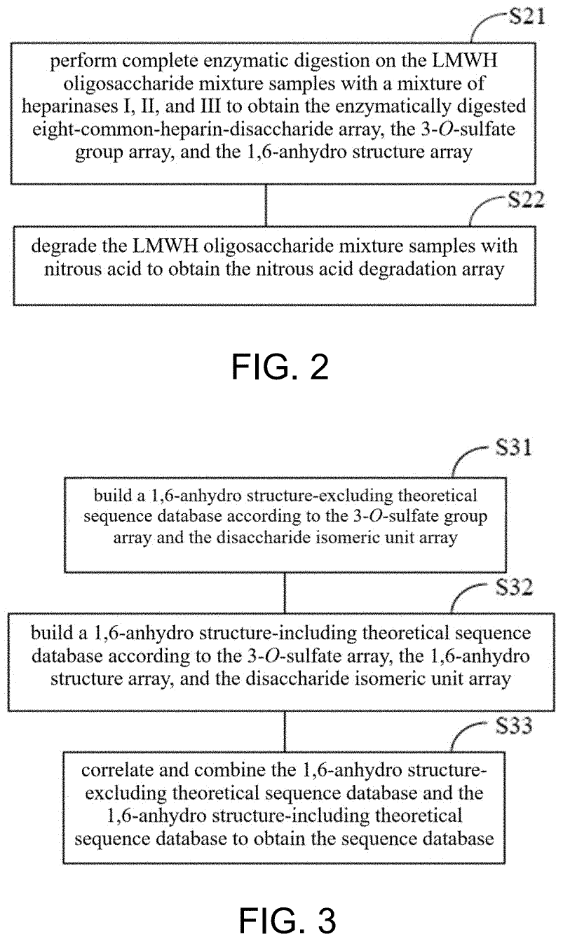

Referring to FIG. 1 to FIG. 3, FIG. 1 is a flowchart of a sequencing method of LMWH oligosaccharides; FIG. 2 is a flowchart of sub-steps of S2 of FIG. 1; and FIG. 3 is a flowchart of sub-steps of S3 of FIG. 1. As shown in FIG. 1 to FIG. 3, the sequencing method of LMWH oligosaccharides of the present disclosure includes:

a sample preparation step S1: a group of LMWH oligosaccharide mixture samples are isolated or prepared according to experimental requirements;

a sample treatment step S2: complete enzymatic digestion and nitrous acid degradation are performed on the LMWH oligosaccharide mixture samples to obtain an enzymatically digested eight-common-heparin-disaccharide array, a 3-O-sulfate group array, a 1,6-anhydro structure array, and a nitrous acid degradation array, respectively;

a data processing step S3: IdoA/GlcA of different disaccharides are calculated according to the eight-common-heparin-disaccharide array and the nitrous acid degradation array to obtain a disaccharide isomeric unit array;

a sequence database building step S4: a sequence database is built according to the degree of polymerization of the oligosaccharide mixture, the disaccharide isomeric unit array, the 3-O-sulfate group array, and the 1,6-anhydro structure array; and

a specific result output step S5: the sequence database is screened according to input qualification information, and then a specific result file is output.

Experimental data are analyzed by auxiliary software for sequencing LMWH drugs. Each oligosaccharide has its unique types and proportions of basic building blocks, which is the theoretical basis of computer-aided structural characterization. Through the content determination of basic building blocks of LMWHs, the probability of appearance of different basic building blocks in an oligosaccharide sequence is obtained. According to the structural law of LMWHs, a possible oligosaccharide sequence database is built through calculation and simulation, and the probability of appearance of a theoretical oligosaccharide sequence can reflect an actual proportion of the oligosaccharide in the oligosaccharide mixture, and thus, sequences and contents of a group of mixed oligosaccharides are obtained. By combining with the existing chromatographic separation technology, the oligosaccharide sequence of each component can be quickly inferred, which can assist scientific researchers in quickly characterizing structures of complex heparin oligosaccharides.

The sample treatment step S2 includes:

an enzymatic digestion treatment step S21: complete enzymatic digestion is performed on the LMWH oligosaccharide mixture samples by using a mixture of heparinases I, II, and III to obtain the enzymatically digested eight-common-heparin-disaccharide array, the 3-O-sulfate group array, and the 1,6-anhydro structure array. Specifically, one heparinase I, one heparinase II, and one heparinase III are taken and mixed; 20 μg/μL of sample solution is prepared, and 2.5 uL of sample solution is taken; 8.75 μL of enzymatic digestion buffer and 12.5 μL of mixed heparinase solution are added, the sample is incubated at 25° C. for 36 hours, added with 12.5 μL of mixed heparinase solution, and incubated for 36 hours; the sample is heated in a water bath at 100° C. for 10 min to inactivate the heparinases, a supernate is collected and freeze-dried; the sample is desalted and freeze-dried to obtain a complete heparinase digestion product; basic building blocks of the sample are determined by a combination technology of high-performance liquid chromatography and high-resolution mass spectrometry (LC-MS) to obtain the enzymatically digested eight-common-heparin-disaccharide array, the 3-O-sulfate group array, and the 1,6-anhydro structure array; and

a nitrous acid degradation step S22: the LMWH oligosaccharide mixture samples are degraded by using nitrous acid to obtain the nitrous acid degradation array. Specifically, 0.5 moL H₂SO₄ and 0.5 moL Ba(NO₂)₂ are taken and placed on ices for pre-cooling; 10 to 15 μg of sample is prepared; 10 μL of pre-cooled 0.5 moL H₂SO₄ and 10 μL of pre-cooled 0.5 moL Ba(NO₂)₂ are taken and mixed uniformly, the mixture is added into and uniformly mixed with the sample by means of vortex, the sample is stood for 10 min; a pH value of the sample is regulated to 4 with 1 moL Na₂CO₃, 20 μL of HONO solution (pH 4, and a ratio of 5.5 moL NaNO₂ to 0.5 moL H₂SO₄ is 5:2) is added into and uniformly mixed with the sample by means of vortex, the sample is stood for 15 min; a pH value of the sample is regulated to 8.5 with 1 moL Na₂CO₃, the sample is reduced with 0.5 moL NaBH₄ at 55° C. for 8 hours; after the reduction is completed, a pH value of the sample is regulated to 4 with 0.1 moL acetic acid and then regulated to 7 with 0.1 moL ammonium hydroxide; the sample is desalted and freeze-dried to obtain a nitrous acid degradation product, basic building blocks of the sample are determined by a combination technology of high-performance liquid chromatography and high-resolution mass spectrometry (LC-MS) to obtain the nitrous acid degradation array.

For example, for the products obtained by performing nitrous acid degradation and complete heparinase digestion on the LMWH sample, the basic building blocks of the sample are determined by a combination technology of high-performance liquid chromatography and high-resolution mass spectrometry (LC-MS).

(1) Analysis of Nitrous Acid Degradation Product

1. High performance liquid chromatography (HPLC) parameters: analytical column: PGC column (Hypercarb Column, 5 um, 150 mm×4.6 mm); mobile phases: A phase: 0.1% formic acid aqueous solution (pH 5.5), and B phase: 90% acetonitrile solution; flow rate: 0.5 mL/min, 100 μL of sample is shunted for mass spectrometry; and elution gradients: 0 to 10 min, 0% B; 10 to 14 min, 0 to 15% B; 14 to 64 min, 15% B; 64.01 to 75 min, 100% B; 75.01 to 90 min, 0% B.

2. Mass spectrometry parameters: m/z scan range: 150 to 1,000; negative ion mode; spray voltage: −4.0 kV; capillary temperature: 275° C.; and resolution: 60,000.

(2) Analysis of Complete Enzymatic Digestion Product

1. High performance liquid chromatography (HPLC) parameters: analytical column: Phenomenex Luna 3 μm HILIC 200 Å (150×2.0 mm); mobile phases: A phase: 5 mmol/L ammonium acetate aqueous solution, and B phase: 5 mmol/L ammonium acetate and 98% acetonitrile solution; flow rate: 0.15 mL/min; and elution gradients: 0-20 min, 95% B; 20-122 min, 95-77% B; 122-127 min, 77-50% B; 127-150 min, 50% B; 150-151 min, 50-95% B; 151-170 min, 95% B.

2. Mass spectrometry parameters: spray voltage: −3.8 kV; m/z scan range: 240 to 800; ion mode: negative ion mode; capillary temperature: 275° C.; and resolution: 60,000.

Through the given information of the basic building blocks obtained by complete enzymatic digestion and nitrous acid degradation and their contents, contents of all the basic building blocks and their isomers are calculated, and all possible sequences and their possibilities are obtained through all arrangements of different basic building blocks. A database of all reasonable theoretical sequences is obtained under the given qualifications, and all possible sequences and their contents are output.

Data of disaccharide units obtained by complete enzymatic digestion and their proportions are defined as an enzymatically digested eight-common-heparin-disaccharide array A and input into software, the enzymatically digested eight-common-heparin-disaccharide array A includes 8 basic building blocks obtained by complete enzymatic digestion, which are ΔIVA, ΔIIIA, ΔIIA, ΔIA, ΔIVS, ΔIIIS, ΔIIS, and ΔIS, respectively. LMWH can be completely degraded into basic building blocks by using the mixture of heparinases I, II, and III, and it also includes 3-O-sulfated tetrasaccharide (of which data are defined as a 3-O-sulfate group array D) and disaccharides and oligosaccharides whose terminals contain a 1,6-anhydro structure (of which data are defined as a 1,6-anhydro structure array E) in addition to the 8 common heparin disaccharides. However, unsaturated double bonds are formed at 4th and 5th sites of uronic acid due to enzymatic digestion, isomeric information of iduronic acid and glucuronic acid is lost, which needs to be completed by nitrous acid degradation.

Data of disaccharide units obtained by nitrous acid degradation and their proportions are defined as a nitrous acid degradation array B and input into the software, and the nitrous acid degradation array B includes: IM, GM, I2SM, G2SM, GM6S, IM6S, I2SM6S, GM3S6S, GM3S, IdoA-GlcNAc(3S/6S)-GlcA-M(3S/6S), G-GlcNAc(6S)-G-M(3,6S), etc. Data of the basic building blocks obtained by nitrous acid degradation include proportions of iduronic acid (IdoA) and glucuronic acid (GlcA) isomers of different disaccharide units, which can supplement the isomeric information lost due to enzymatic digestion.

It should be noted that the nitrous acid degradation array B actually includes more basic building blocks, if there are more components, users can add them according to the experimental results. In the present embodiment, the main components are used to calculate IdoA/GlcA proportions and generate a disaccharide isomeric unit array C.

The disaccharide isomeric unit array C includes: IdoA-GlcNAc, GlcA-GlcNAc, IdoA2S-GlcNAc, GlcA2S-GlcNAc, IdoA-GlcNAc6S, GlcA-GlcNAc6S, IdoA2S-GlcNAc6S, GlcA2S-GlcNAc6S, IdoA-GlcNS, GlcA-GlcNS, IdoA2S-GlcNS, GlcA2S-GlcNS, IdoA-GlcNS6S, GlcA-GlcNS6S, IdoA2S-GlcNS6S, and GlcA2S-GlcNS6S. Values of this array are calculated based on the array A and the array B.

Specifically, the values of the array C are calculated based on the array A and the array B according to the following formulas:

IdoA-GlcNAc=ΔIVA*IM/(IM+GM)

GlcA-GlcNAc=ΔIVA*GM/(IM+GM)

IdoA2S-GlcNAc=ΔIIIA*I2SM/(I2SM+G2SM)

GlcA2S-GlcNAc=ΔIIIA*G2SM/(I2SM+G2SM)

IdoA-GlcNAc6S=ΔIIA*IM6S/(IM6S+GM6S)

GlcA-GlcNAc6S=ΔIIA*GM6S/(IM6S+GM6S)

IdoA2S-GlcNAc6SΔ=IA*I2SM6S/(I2SM6S+GM3S6S)

GlcA2S-GlcNAc6S=ΔIA*GM3S6S/(I2SM6S+GM3S6S)

IdoA-GlcNS=ΔIVS*IM/(IM+GM)

GlcA-GlcNS=ΔIVS*GM/(IM+GM)

IdoA2S-GlcNS=ΔIIIS*I2SM/(I2SM+G2SM)

GlcA2S-GlcNS=ΔIIIS*G2SM/(I2SM+G2SM)

IdoA-GlcNS6S=ΔIIS*IM6S/(IM6S+GM6S)

GlcA-GlcNS6S=ΔIIS*GM6S/(IM6S+GM6S)

IdoA2S-GlcNS6S=ΔIS*I2SM6S/(I2SM6S+G2SM6S) (the default value is 1)

GlcA2S-GlcNS6S=ΔIS*G2SM6S/(I2SM6S+G2SM6S) (the default value is 0)

The degree of polymerization of the oligosaccharide mixture, and special structures, such as 1,6-anhydro (i.e. the 1,6-anhydro structure array E), 3-O-sulfated tetrasaccharide (i.e. the 3-O-sulfate group array D), and saturated terminal structures, of LMWH and their proportions are input into the software. Calculation methods of sequences containing 1,6-anhydro and 3-O-sulfated tetrasaccharide are described herein. It should be noted that LMWHs have many types of special terminal structures among which main components are disclosed herein, and contents of other components are relatively low. In practical experiments, if other terminal structures are detected, a calculation method similar to that of 1,6-anhydro (i.e. the 1,6-anhydro structure array) is used, that is, the terminal structure is fixed to an end of a sequence, the rest of the sequence is generated in the same way, which will not be described in detail herein.

The 3-O-sulfate group array D: the structural basis of the anticoagulant activity of LMWHs is a class of pentasaccharide sequences containing 3-O-sulfate groups, which will generate unsaturated tetrasaccharides containing 3-O-sulfate groups after being completely digested by heparinase. These basic building blocks constitute the 3-O-sulfate group array D: IdoA-GlcNAc6S-GlcA-GlcNS3 S6S, IdoA-GlcNS6S-GlcA-GlcNS3 S6S, IdoA-GlcNAc6S-GlcA-GlcNS3S, IdoA2S-GlcNAc6S-GlcA-GlcNS3S6S, and IdoA2S-GlcNS6S-GlcA-GlcNS3S, and values are assigned to the array by input.

The 1,6-anhydro structure array E: refers to special terminal structures of Enoxaparin sodium of LMWHs. These basic building blocks constitute the 1,6-anhydro structure array E: IdoA2S-GlcNS6S-IdoA2S-ManNS-1,6-anhydro, GlcA-GlcNS/ManNS-1,6-anhydro, and IdoA2S-GlcNS-1,6-anhydro, and values are assigned to the array by input.

The degree of polymerization of an oligosaccharide mixture dp: refers to the length of the oligosaccharide mixture, i.e. the number of monosaccharides that make up the oligosaccharide mixture. Variates such as the degree of polymerization of an oligosaccharide mixture dp, the number Number, a 1,6-anhydro structure-excluding coefficient d1, a proportion of normal sequences Percent_normal, a proportion of sequences with GlcA-GlcNS/ManNS-1,6-anhydro at the right terminal Percent_GlcA-GlcNS/ManNS-1,6-anhydro, a proportion of sequences with IdoA2S-GlcNS-1,6-anhydro at the right terminal Percent_IdoA2S-GlcNS-1,6-anhydro, and a proportion of sequences with IdoA2S-GlcNS6S-IdoA2S-ManNS-1,6-anhydro at the right terminal Percent_IdoA2S-GlcNS6S-IdoA2S-ManNS-1,6-anhydro are defined, and a value is assigned to the degree of polymerization of the oligosaccharide mixture by input.

Further, the sequence database building step S3 includes:

a 1,6-anhydro structure-excluding theoretical sequence database building step S31: a 1,6-anhydro structure-excluding theoretical sequence database is built according to the 3-O-sulfate group array and the disaccharide isomeric unit array. The 1,6-anhydro structure-excluding theoretical sequence database building step includes: if all elements in the 3-O-sulfate group array are 0, the number of selected elements is set as dp/2, dp/2 elements are selected from the disaccharide isomeric unit array, and all the selected elements are arranged in a row in order, string concatenation of various elements and a product of values of various elements are calculated and output; and if all elements in the 3-O-sulfate group array are not 0, the number of selected elements is set as (dp−4)/2, at least one element is selected from the 3-O-sulfate group array, (dp−4)/2 elements are selected from the disaccharide isomeric unit array, the remaining selected elements are arranged in a row in order, and string concatenation of various elements and a product of values of various elements are calculated and output, wherein dp is the degree of polymerization of the oligosaccharide mixture,

specifically, in one embodiment of the present disclosure, generally, if dp is less than 10, the default setting is to select one element from the 3-O-sulfate group array. In one embodiment of the present disclosure, if a user has special requirements for the number of elements selected from 3-O-sulfate group array, a plurality of elements can also be selected from the 3-O-sulfate group array, and the same element can be selected repeatedly. If the number of selected elements is n, (dp−4n)/2 elements are subsequently selected from the disaccharide isomeric unit array;

a 1,6-anhydro structure-including theoretical sequence database building step S32: a 1,6-anhydro structure-including theoretical sequence database is built according to the 3-O-sulfate group array, the 1,6-anhydro structure array, and the disaccharide isomeric unit array. The 1,6-anhydro structure-including theoretical sequence database building step includes: if an IdoA2S-GlcNS6S-IdoA2S-ManNS-1,6-anhydro group in the 1,6-anhydro structure array is greater than 0 and all elements in the 3-O-sulfate group array are 0, the number of selected elements is set as (dp−4)/2, one element (IdoA2S-GlcNS6S-IdoA2S-ManNS-1,6-anhydro) in the 1,6-anhydro structure array is selected, (dp−4)/2 elements are selected from the disaccharide isomeric unit array, the element (IdoA2S-GlcNS6S-IdoA2S-ManNS-1,6-anhydro) is set as the rightmost element of a string, the (dp−4)/2 elements that are selected from the disaccharide isomeric unit array are arranged in a row in order on the left side of the element, and string concatenation of various elements and a product of values of various elements are calculated and output; if an IdoA2S-GlcNS6S-IdoA2S-ManNS-1,6-anhydro group in the 1,6-anhydro structure array is greater than 0 and all elements in the 3-O-sulfate group array are not 0, the number of selected elements is set as (dp−4−4)/2, one element is selected from the 3-O-sulfate group array, one element (IdoA2S-GlcNS6S-IdoA2S-ManNS-1,6-anhydro) is selected from the 1,6-anhydro structure array, (dp−4−4)/2 elements are selected from the disaccharide isomeric unit array, IdoA2S-GlcNS6S-IdoA2S-ManNS-1,6-anhydro element is set as the rightmost element of a string, the remaining selected elements are arranged in a row in order, and string concatenation of various elements and a product of values of various elements are calculated and output; if a GlcA-GlcNS/ManNS-1,6-anhydro group or an IdoA2S-GlcNS-1,6-anhydro group in the 1,6-anhydro structure array is greater than 0 and all elements in the 3-O-sulfate group array are 0, the number of selected elements is set as (dp−2)/2, one element (GlcA-GlcNS/ManNS-1,6-anhydro or IdoA2S-GlcNS-1,6-anhydro) is selected from the 1,6-anhydro structure array, (dp−2)/2 elements are selected from the disaccharide isomeric unit array, GlcA-GlcNS/ManNS-1,6-anhydro or IdoA2S-GlcNS-1,6-anhydro is set as the rightmost element of a string, the (dp−2)/2 elements that are selected from the disaccharide isomeric unit array are arranged in a row in order on the left side of the element, and string concatenation of various elements and a product of values of various elements are calculated and output; and if a GlcA-GlcNS/ManNS-1,6-anhydro group or an IdoA2S-GlcNS-1,6-anhydro group in the 1,6-anhydro structure array is greater than 0 and all elements in the 3-O-sulfate group array are not 0, the number of selected elements is set as (dp−4−2)/2, one element (IdoA-GlcNAc6S-GlcA-GlcNS3S6S or IdoA-GlcNS6S-GlcA-GlcNS3S6S or IdoA-GlcNAc6S-GlcA-GlcNS3S or IdoA2S-GlcNAc6S-GlcA-GlcNS3S6S or IdoA2S-GlcNS6S-GlcA-GlcNS3S) is selected from the 3-O-sulfate group array, one element (GlcA-GlcNS/ManNS-1,6-anhydro or IdoA2S-GlcNS-1,6-anhydro) is selected from the 1,6-anhydro structure array, (dp−4−2)/2 elements are selected from the disaccharide isomeric unit array, GlcA-GlcNS/ManNS-1,6-anhydro or IdoA2S-GlcNS-1,6-anhydro is set as the rightmost element of a string, the remaining selected elements are arranged in a row in order, and string concatenation of various elements and a product of values of various elements are calculated and output; and

a combination step S33: the 1,6-anhydro structure-excluding theoretical sequence database and the 1,6-anhydro structure-including theoretical sequence database are correlated and combined to obtain the sequence database. The combination step S33 includes: results of the 1,6-anhydro structure-excluding theoretical sequence database and results of the 1,6-anhydro structure-including theoretical sequence database are combined and sorted according the value to obtain the sequence database.

Specifically, the sequence database is formed by combining (1) the 1,6-anhydro structure-excluding theoretical sequence database with (2) the 1,6-anhydro structure-including theoretical sequence database, and the two sequence databases are combined to form a complete theoretical sequence database. The two parts are respectively generated as follows:

before calculation, proportional coefficients are set, which are correction coefficients of different types of sequences, and input by an experimenter according to experimental results to correct contents of different types of sequences. The data type is integer or decimal, and the software default value is 1.

# Proportion of Normal Sequences (Manual Input)

Percent_normal=[1.0]

# Proportion of Sequences with IdoA2S-GlcNS6S-IdoA2S-ManNS-1,6-Anhydro at the Right Terminal E[0] (Manual Input)

Percent_E0=[1.0]

# Proportion of Sequences with GlcA-GlcNS/ManNS-1,6-Anhydro at the Right Terminal E[1] (Manual Input)

Percent_E1=[1.0]

# Proportion of Sequences with IdoA2S-GlcNS-1,6-Anhydro at the Right Terminal E[2] (Manual Input)

Percent_E2=[1.0]

(1) 1,6-anhydro Structure-Excluding Theoretical Sequence Database

If all elements in the 3-O-sulfate group array are 0,

then, Number=dp/2, Number elements are selected from the disaccharide isomeric unit array C, the same element can be selected repeatedly, the selected elements are arranged in a row in order, string concatenation of various elements and a product of values of various elements are calculated and output, and all the results are multiplied by Percent_normal.

Example: if the array C includes only three elements, i.e. IdoA-GlcNAc, GlcA-GlcNAc, and IdoA2S-GlcNAc, whose values are 1, 2, and 3, respectively, values of all elements in the 1,6-anhydro structure array E are 0, and Percent_normal=1.

If dp=4, then Number=2, and output results are as follows:

[IdoA-GlcNAc-IdoA-GlcNAc]=1*1*Percent_normal=1

[IdoA-GlcNAc-GlcA-GlcNAc]=1*2*Percent_normal=2

[IdoA-GlcNAc-IdoA2S-GlcNAc]=1*3*Percent_normal=3

[GlcA-GlcNAc-IdoA-GlcNAc]=2*1*Percent_normal=2

[GlcA-GlcNAc-GlcA-GlcNAc]=2*2*Percent_normal=4

[GlcA-GlcNAc-IdoA2S-GlcNAc]=2*3*Percent_normal=6

[IdoA2S-GlcNAc-IdoA-GlcNAc]=3*1*Percent_normal=3

[IdoA2S-GlcNAc-GlcA-GlcNAc]=3*2*Percent_normal=6

[IdoA2S-GlcNAc-IdoA2S-GlcNAc]=3*3*Percent_normal=9

Otherwise, Number=(dp−4)/2, one element (IdoA-GlcNAc6S-GlcA-GlcNS3S6S or IdoA-GlcNS6S-GlcA-GlcNS3S6S or IdoA-GlcNAc6S-GlcA-GlcNS3S or IdoA2S-GlcNAc6S-GlcA-GlcNS3S6S, or IdoA2S-GlcNS6S-GlcA-GlcNS3S) is selected from the 3-O-sulfate group array D, Number elements are selected from the disaccharide isomeric unit array C, and the same element can be selected repeatedly. The Number+1 selected elements are arranged in a row in order. String concatenation of various elements and a product of values of various elements are output, and all the product results are multiplied by Percent_normal.

Example: if the array C includes only two elements, i.e. IdoA-GlcNAc and GlcA-GlcNAc, whose values are 1 and 2, respectively; the 3-O-sulfate group array D includes only IdoA-GlcNAc6S-GlcA-GlcNS3S6S, whose value is not 0 but 3; and values of all elements in the 1,6-anhydro structure array E are 0.

If dp=8, then Number=2, and output results are as follows:

[IdoA-GlcNAc6S-GlcA-GlcNS3S6S-IdoA-GlcNAc-IdoA-GlcNAc]=3*1*1*Percent_normal=3

[IdoA-GlcNAc-IdoA-GlcNAc6S-GlcA-GlcNS3S6S-IdoA-GlcNAc]=1*3*1*Percent_normal=3

[IdoA-GlcNAc-IdoA-GlcNAc-IdoA-GlcNAc6S-GlcA-GlcNS3S6S]=1*1*3*Percent_normal=3

[IdoA-GlcNAc6S-GlcA-GlcNS3S6S-IdoA-GlcNAc-GlcA-GlcNAc]=3*1*2*Percent_normal=6

[IdoA-GlcNAc-IdoA-GlcNAc6S-GlcA-GlcNS3S6S-GlcA-GlcNAc]=1*3*2*Percent_normal=6

[IdoA-GlcNAc-GlcA-GlcNAc-IdoA-GlcNAc6S-GlcA-GlcNS3S6S]=1*2*3*Percent_normal=6

[IdoA-GlcNAc6S-GlcA-GlcNS3S6S-GlcA-GlcNAc-IdoA-GlcNAc]=3*2*1*Percent_normal=6

[GlcA-GlcNAc-IdoA-GlcNAc6S-GlcA-GlcNS3S6S-IdoA-GlcNAc]=2*3*1*Percent_normal=6

[GlcA-GlcNAc-IdoA-GlcNAc-IdoA-GlcNAc6S-GlcA-GlcNS3S6S]=2*1*3*Percent_normal=6

[IdoA-GlcNAc6S-GlcA-GlcNS3 S6S-GlcA-GlcNAc-GlcA-GlcNAc]=3*2*2*Percent_normal=12

[GlcA-GlcNAc-IdoA-GlcNAc6S-GlcA-GlcNS3S6S-GlcA-GlcNAc]=2*3*2*Percent_normal=12

[GlcA-GlcNAc-GlcA-GlcNAc-IdoA-GlcNAc6S-GlcA-GlcNS3S6S]=2*2*3*Percent_normal=12

. . .

(2) The 1,6-anhydro structure-including theoretical sequence database is formed by combining a sequence database of an IdoA2S-GlcNS6S-IdoA2S-ManNS-1,6-anhydro group with a sequence database of a GlcA-GlcNS/ManNS-1,6-anhydro and IdoA2S-GlcNS-1,6-anhydro group, the sequence databases of the two groups are generated as follows:

1. The IdoA2S-GlcNS6S-IdoA2S-ManNS-1,6-anhydro group, judgment criteria: if IdoA2S-GlcNS6S-IdoA2S-ManNS-1,6-anhydro is greater than 0, then the following steps are performed, and if IdoA2S-GlcNS6S-IdoA2S-ManNS-1,6-anhydro is equal to 0, then the following steps are not performed.

If all elements in the 3-O-sulfate group array D are 0,

then, Number=(dp−4)/2, the element (IdoA2S-GlcNS6S-IdoA2S-ManNS-1,6-anhydro) is selected from the 1,6-anhydro structure array E, Number elements are selected from the disaccharide isomeric unit array C, and the same element can be selected repeatedly.

IdoA2S-GlcNS6S-IdoA2S-ManNS-1,6-anhydro is set as the rightmost element of a string, and the Number elements that are selected from the disaccharide isomeric unit array C are arranged in a row in order on the left side of the element. String concatenation of various elements and a product of values of various elements are output, and all the products are multiplied by Percent_IdoA2S-GlcNS6S-IdoA2S-ManNS-1,6-anhydro.

### Example

[IdoA-GlcNAc- . . . GlcA-GlcNAc-IdoA2S-GlcNS6S-IdoA2S-ManNS-1,6-anhydro]=a product of values of various elements*Percent_IdoA2S-GlcNS6S-IdoA2S-ManNS-1,6-anhydro

[GlcA-GlcNAc- . . . IdoA-GlcNAc-IdoA2S-GlcNS6S-IdoA2S-ManNS-1,6-anhydro]=a product of values of various elements*Percent_IdoA2S-GlcNS6S-IdoA2S-ManNS-1,6-anhydro

[GlcA-GlcNAc- . . . GlcA-GlcNAc-IdoA2S-GlcNS6S-IdoA2S-ManNS-1,6-anhydro]=a product of values of various elements*Percent_IdoA2S-GlcNS6S-IdoA2S-ManNS-1,6-anhydro

[IdoA-GlcNAc- . . . IdoA-GlcNAc-IdoA2S-GlcNS6S-IdoA2S-ManNS-1,6-anhydro]=a product of values of various elements*Percent_IdoA2S-GlcNS6S-IdoA2S-ManNS-1,6-anhydro

. . .

Otherwise, Number=(dp−4−4)/2, one element (IdoA-GlcNAc6S-GlcA-GlcNS3S6S or IdoA-GlcNS6S-GlcA-GlcNS3S6S or IdoA-GlcNAc6S-GlcA-GlcNS3S or IdoA2S-GlcNAc6S-GlcA-GlcNS3S6S or IdoA2S-GlcNS6S-GlcA-GlcNS3S) is selected from the 3-O-sulfate group array D; the element (IdoA2S-GlcNS6S-IdoA2S-ManNS-1,6-anhydro) is selected from the 1,6-anhydro structure array E; Number elements are selected from the disaccharide isomeric unit array C, and the same element can be selected repeatedly.

IdoA2S-GlcNS6S-IdoA2S-ManNS-1,6-anhydro is set as the rightmost element of a string, and the remaining Number+1 elements are arranged in a row in order. String concatenation of various elements and a product of values of various elements are output, and all the products are multiplied by Percent_IdoA2S-GlcNS6S-IdoA2S-ManNS-1,6-anhydro.

### Example

[IdoA-GlcNAc-IdoA-GlcNAc6S-GlcA-GlcNS3S6S-IdoA-GlcNAc-IdoA2S-GlcNS6S-IdoA2S-ManNS-1,6-anhydro]=a product of values of various elements*Percent_IdoA2S-GlcNS6S-IdoA2S-ManNS-1,6-anhydro

[IdoA-GlcNAc6S-GlcA-GlcNS3 S6S-IdoA-GlcNAc-IdoA-GlcNAc-IdoA2S-GlcNS6S-IdoA2S-ManNS-1,6-anhydro]=a product of values of various elements*Percent_IdoA2S-GlcNS6S-IdoA2S-ManNS-1,6-anhydro

[IdoA-GlcNAc-IdoA-GlcNAc-IdoA-GlcNAc6S-GlcA-GlcNS3S6S-IdoA2S-GlcNS6S-IdoA2S-ManNS-1,6-anhydro]=a product of values of various elements*Percent_IdoA2S-GlcNS6S-IdoA2S-ManNS-1,6-anhydro

[IdoA-GlcNAc-IdoA-GlcNAc6S-GlcA-GlcNS3S6S-GlcA-GlcNAc-IdoA2S-GlcNS6S-IdoA2S-ManNS-1,6-anhydro]=a product of values of various elements*Percent_IdoA2S-GlcNS6S-IdoA2S-ManNS-1,6-anhydro

[IdoA-GlcNAc6S-GlcA-GlcNS3S6S-GlcA-GlcNAc-IdoA-GlcNAc-IdoA2S-GlcNS6S-IdoA2S-ManNS-1,6-anhydro]=a product of values of various elements*Percent_IdoA2S-GlcNS6S-IdoA2S-ManNS-1,6-anhydro

[GlcA-GlcNAc-IdoA-GlcNAc-IdoA-GlcNAc6S-GlcA-GlcNS3S6S-IdoA2S-GlcNS6S-IdoA2S-ManNS-1,6-anhydro]=a product of values of various elements*Percent_IdoA2S-GlcNS6S-IdoA2S-ManNS-1,6-anhydro

. . .

2. The GlcA-GlcNS/ManNS-1,6-anhydro and IdoA2S-GlcNS-1,6-anhydro group, judgment criteria: if GlcA-GlcNS/ManNS-1,6-anhydro or IdoA2S-GlcNS-1,6-anhydro is greater than 0, then the following steps are performed, and if GlcA-GlcNS/ManNS-1,6-anhydro is equal to 0 and IdoA2S-GlcNS-1,6-anhydro is equal to 0, the following steps are not performed.

If all elements in the 3-O-sulfate group array D are 0,

then, Number=(dp−2)/2, one element (GlcA-GlcNS/ManNS-1,6-anhydro or IdoA2S-GlcNS-1,6-anhydro) is selected from the 1,6-anhydro structure array E, Number elements are selected from the disaccharide isomeric unit array C, and the same element can be selected repeatedly.

GlcA-GlcNS/ManNS-1,6-anhydro or IdoA2S-GlcNS-1,6-anhydro is set as the rightmost element of a string, the Number elements that are selected from the disaccharide isomeric unit array C are arranged in a row in order on the left side of the element. String concatenation of various elements and a product of values of various elements are output, and various products are respectively multiplied by Percent_GlcA-GlcNS/ManNS-1,6-anhydro or Percent_IdoA2S-GlcNS-1,6-anhydro (based on the rightmost element).

### Example

[IdoA-GlcNAc- . . . GlcA-GlcNAc-GlcA-GlcNS/ManNS-1,6-anhydro]=a product of values of various elements*Percent_GlcA-GlcNS/ManNS-1,6-anhydro

[GlcA-GlcNAc- . . . IdoA-GlcNAc-GlcA-GlcNS/ManNS-1,6-anhydro]=a product of values of various elements*Percent_GlcA-GlcNS/ManNS-1,6-anhydro

[GlcA-GlcNAc- . . . GlcA-GlcNAc-GlcA-GlcNS/ManNS-1,6-anhydro]=a product of values of various elements*Percent_GlcA-GlcNS/ManNS-1,6-anhydro

[IdoA-GlcNAc- . . . IdoA-GlcNAc-GlcA-GlcNS/ManNS-1,6-anhydro]=a product of values of various elements*Percent_GlcA-GlcNS/ManNS-1,6-anhydro

. . .

[IdoA-GlcNAc- . . . GlcA-GlcNAc-IdoA2S-GlcNS-1,6-anhydro]=a product of values of various elements*Percent_IdoA2S-GlcNS-1,6-anhydro

[GlcA-GlcNAc- . . . IdoA-GlcNAc-IdoA2S-GlcNS-1,6-anhydro]=a product of values of various elements*Percent_IdoA2S-GlcNS-1,6-anhydro

[GlcA-GlcNAc- . . . GlcA-GlcNAc-IdoA2S-GlcNS-1,6-anhydro]=a product of values of various elements*Percent_IdoA2S-GlcNS-1,6-anhydro

[IdoA-GlcNAc- . . . IdoA-GlcNAc-IdoA2S-GlcNS-1,6-anhydro]=a product of values of various elements*Percent_IdoA2S-GlcNS-1,6-anhydro

. . .

Otherwise, Number=(dp−4−2)/2, one element (IdoA-GlcNAc6S-GlcA-GlcNS3S6S or IdoA-GlcNS6S-GlcA-GlcNS3S6S or IdoA-GlcNAc6S-GlcA-GlcNS3S or IdoA2S-GlcNAc6S-GlcA-GlcNS3S6S or IdoA2S-GlcNS6S-GlcA-GlcNS3S) is selected from the 3-O-sulfate group array D; one element (GlcA-GlcNS/ManNS-1,6-anhydro or IdoA2S-GlcNS-1,6-anhydro) is selected from the 1,6-anhydro structure array E; Number elements are selected from the disaccharide isomeric unit array C, and the same element can be selected repeatedly.

GlcA-GlcNS/ManNS-1,6-anhydro or IdoA2S-GlcNS-1,6-anhydro is set as the rightmost element of a string, the remaining Number+1 elements are arranged in a row in order. String concatenation of various elements and a product of values of various elements are output, and various products are respectively multiplied by Percent_GlcA-GlcNS/ManNS-1,6-anhydro or Percent_IdoA2S-GlcNS-1,6-anhydro (based on the rightmost element).

### Example

[IdoA-GlcNAc-IdoA-GlcNAc6S-GlcA-GlcNS3S6S-IdoA-GlcNAc-GlcA-GlcNS/ManNS-1,6-anhydro]=a product of values of various elements*Percent_GlcA-GlcNS/ManNS-1,6-anhydro

[IdoA-GlcNAc6S-GlcA-GlcNS3S6S-IdoA-GlcNAc-IdoA-GlcNAc-GlcA-GlcNS/ManNS-1,6-anhydro]=a product of values of various elements*Percent_GlcA-GlcNS/ManNS-1,6-anhydro

[IdoA-GlcNAc-IdoA-GlcNAc-IdoA-GlcNAc6S-GlcA-GlcNS3S6S-GlcA-GlcNS/ManNS-1,6-anhydro]=a product of values of various elements*Percent_IdoA2S-GlcNS-1,6-anhydro

. . .

[IdoA-GlcNAc-IdoA-GlcNAc6S-GlcA-GlcNS3S6S-IdoA-GlcNAc-IdoA2S-GlcNS-1,6-anhydro]=a product of values of various elements*Percent_IdoA2S-GlcNS-1,6-anhydro

[IdoA-GlcNAc6S-GlcA-GlcNS3S6S-IdoA-GlcNAc-IdoA-GlcNAc-IdoA2S-GlcNS-1,6-anhydro]=a product of values of various elements*Percent_IdoA2S-GlcNS-1,6-anhydro

[IdoA-GlcNAc-IdoA-GlcNAc-IdoA-GlcNAc6S-GlcA-GlcNS3S6S-IdoA2S-GlcNS-1,6-anhydro]=a product of values of various elements*Percent_IdoA2S-GlcNS-1,6-anhydro

. . .

Finally, results of the first group of 1,6-anhydro structure-excluding sequences and results of the second group of 1,6-anhydro structure-including sequences are combined, sorted according to the value from high to low, and input into a basic theoretical sequence database file to obtain the sequence database.

It should be noted that in one embodiment of the present disclosure, the multiplication operation of the proportion coefficients, that is, default proportion coefficients are all 1.

More further, the result file includes: 1,6-anhydro structure-including sequences—generation time, 1,6-anhydro structure-excluding sequences—generation time, total sequences—sorted in decreasing order based on the content—generation time, selected sequences—at least one of generation time and a log file that includes at least one of the total number of calculated sequences, the type and value of the disaccharide isomeric unit array, the total record number of total sequences, the total record number of 1,6-anhydro structure-excluding sequences, and the total record number of 1,6-anhydro structure-including sequences.

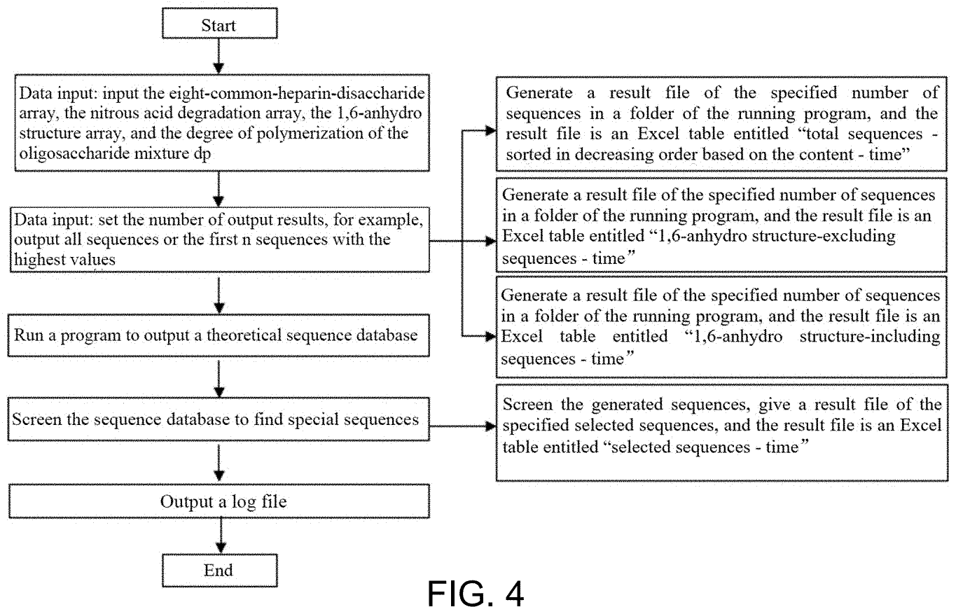

Specifically, referring to FIG. 4, the sequence database is screened according to input qualification information and a specific result file is output, the qualification information includes: the set number of output results, for example, all sequences are output, or the first n sequences with the highest values and selected sequences are output, and the result file includes: 1,6-anhydro structure-including sequences—generation time, 1,6-anhydro structure-excluding sequences—generation time, total sequences—sorted in decreasing order based on the content—generation time, selected sequences—at least one of generation time and a log file that includes at least one of the total number of calculated sequences, the type and value of the disaccharide isomeric unit array, the total record number of total sequences, the total record number of 1,6-anhydro structure-excluding sequences, and the total record number of 1,6-anhydro structure-including sequences.

For example, A: total sequences

(1) The total number of calculated sequences is counted and output to a log file. For example, a total of 50,000 sequences are generated, and an output is that: a generated theoretical sequence database includes 50,000 sequences.

(2) The sequences are sorted in decreasing order, the first q sequences are selected, and an Excel table is output. (q is set by a user, if the number of the sequences is less than q, then all the sequences are shown).

B: After 1,6-anhydro structure-excluding sequences are generated, an Excel table of the 1,6-anhydro structure-excluding sequences is output separately, the sequences are sorted in decreasing order based on the content, and the first n sequences are selected. (n is set by a user, if the number of the sequences is less than 0, then all the sequences are shown).

C: After 1,6-anhydro structure-including sequences are generated, an Excel table of the 1,6-anhydro structure-excluding sequences is output separately. The sequences are sorted in decreasing order based on the content, and the first m sequences are selected. (m is set by a user, if the number of the sequences is less than m, then all the sequences are shown).

D: When sequences are selected, the selected sequences are sorted in decreasing order based on the content, and the first p sequences are output to an Excel table. Each file may be named with time plus selection conditions. (p is set by a user, if the number of the sequences is less than p, then all the sequences are shown).

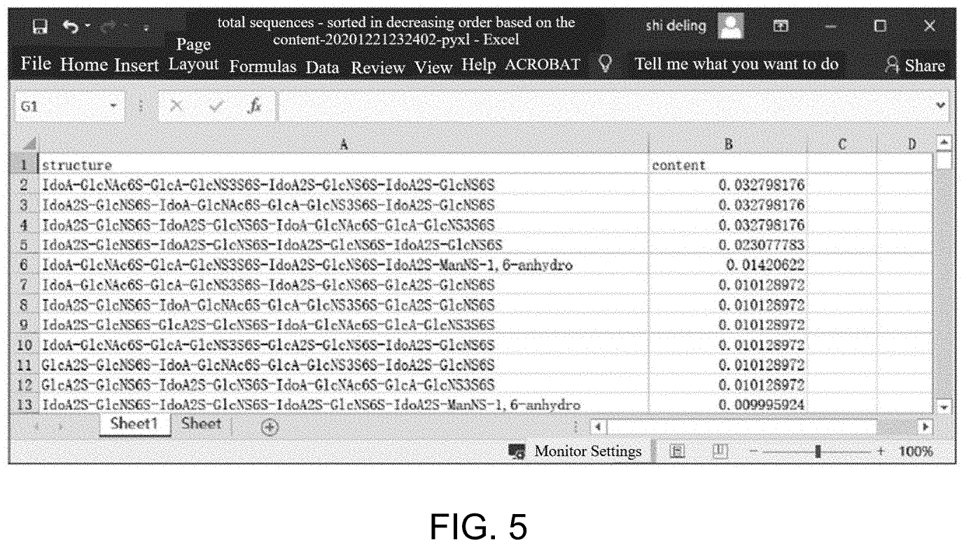

Referring to FIG. 5, specifically, the format of the result file is that: the first column is a sequence, and the second column is the content of the sequence, that is, a product of values of corresponding building blocks.

In one embodiment of the present disclosure, the sequence database is screened, selection conditions are input, and a result is selected from the sequence database. Example of selection conditions for anticoagulant heparin oligosaccharides: results containing IdoA-GlcNAc6S-GlcA-GlcNS3S6S-IdoA2S-GlcNS (6S), IdoA-GlcNS6S-GlcA-GlcNS3S6S-IdoA2S-GlcNS (6S), and

IdoA-GlcNS6S-GlcA-GlcNS3S-IdoA2S-GlcNS (6S) are selected, and a result file is output as a special theoretical sequence database.

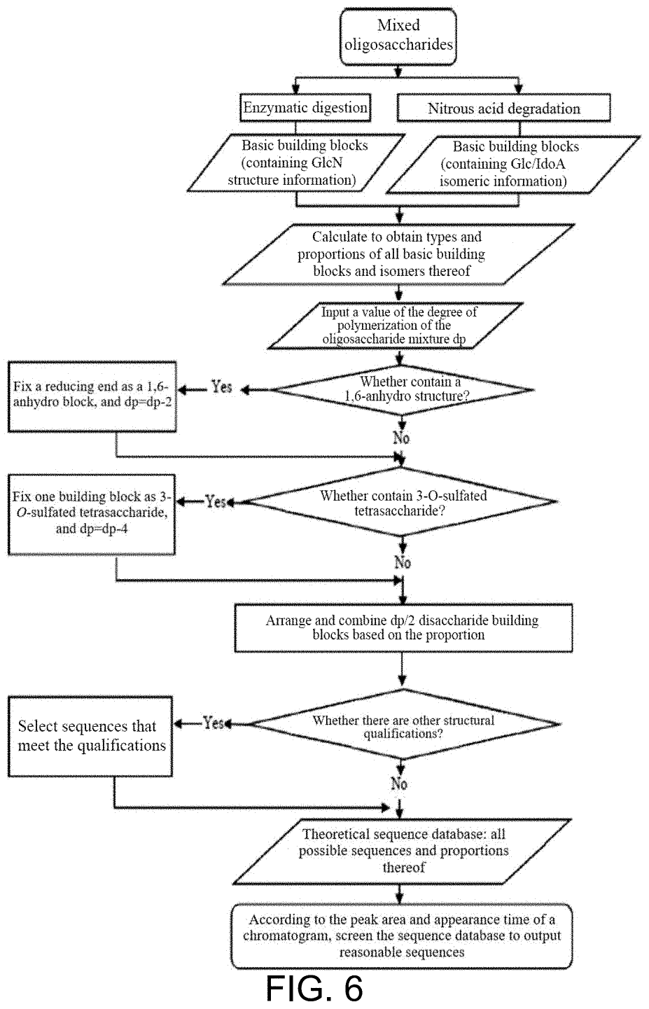

Referring to FIG. 6, which is an application process diagram of the present disclosure. As shown in FIG. 6, the sequencing method of the present disclosure includes:

1. A group of LMWH oligosaccharide mixture samples are isolated or prepared according to experimental requirements.

2. Complete enzymatic digestion is performed on the samples by using a mixture of heparinases I, II, and III to obtain enzymatically digested basic building blocks and proportions thereof, and these data are defined as an enzymatically digested eight-common-heparin-disaccharide array A and input into software.

3. Nitrous acid degradation is performed on the samples to obtain all degraded basic building blocks and proportions thereof, and these data are defined as a nitrous acid degradation array B and input into software.

4. The data input at steps 2 and 3 are processed by the software, IdoA/GlcA of different disaccharides is calculated by the software to obtain types and proportions of all basic building blocks and isomers thereof, and these data are defined as a disaccharide isomeric unit array C and used as the basis for building a sequence database.

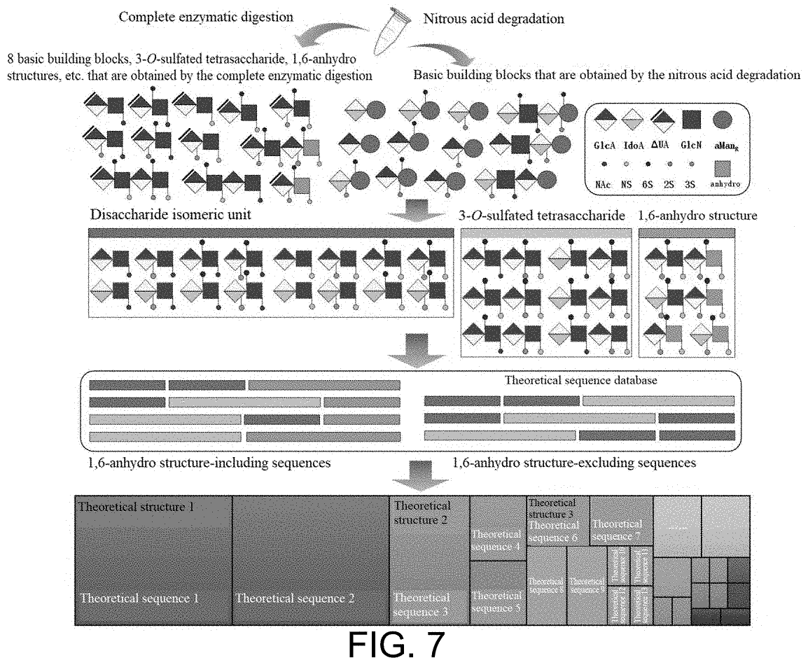

5. The degree of polymerization of the oligosaccharide mixture (according to the experimental requirements), proportions of the basic building blocks and isomers thereof, and special structures, such as 1,6-anhydro (corresponding to a 1,6-anhydro structure array E) and 3-0-sulfated tetrasaccharide (corresponding to a 3-O-sulfate group array D), of LMWH and their proportions are input into In Silico Sequencing (ISS) software to build an initial sequence database. The sequence database building process is shown as FIG. 7.

6. In case of special sequence requirements, sequence qualifications are input, sequences that meet the requirements are selected, and a theoretical sequence database including all the sequences that meet the requirements and their proportions is built.

7. By comparing the sequence database with the order of appearance and contents of components obtained by the existing separation method, sequences of various components can be inferred, which can assist scientific researchers in characterizing sequences of a group of complex oligosaccharide mixtures.

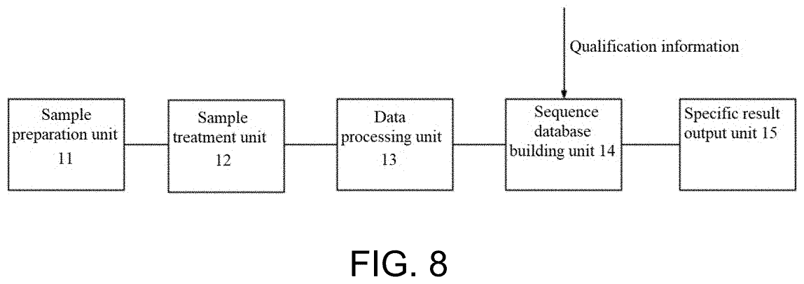

Referring to FIG. 8, which is a schematic structural diagram of a sequencing system of LMWH oligosaccharides. As shown in FIG. 8, the sequencing system of LMWH oligosaccharides includes:

a sample preparation unit 11, configured to isolate or prepare a group of LMWH oligosaccharide mixture samples according to experimental requirements;

a sample treatment unit 12, configured to perform complete enzymatic digestion and nitrous acid degradation on the LMWH oligosaccharide mixture samples to obtain an enzymatically digested eight-common-heparin-disaccharide array, a 3-O-sulfate group array, a 1,6-anhydro structure array, and a nitrous acid degradation array, respectively;

a data processing unit 13, configured to calculate IdoA/GlcA of different disaccharides according to the eight-common-heparin-disaccharide array and the nitrous acid degradation array to obtain a disaccharide isomeric unit array;

a sequence database building unit 14, configured to build a sequence database according to the degree of polymerization of the oligosaccharide mixture, the disaccharide isomeric unit array, the 3-O-sulfate group array, and the 1,6-anhydro structure array; and

a specific result output unit 15, configured to screen the sequence database according to input qualification information, and then output a specific result file.

Based on the above, the present disclosure has the following advantages:

1. At present, commonly used analysis methods for LMWH drugs include the analysis of basic building blocks, and generally enzymatic digestion is the main method. However, at present, there is no kit for sequencing basic building blocks of LMWH drugs. The kit of the present disclosure can provide scientific researchers and R&D staffs of enterprises with a simple and rapid sequencing mean. The kit can be used to simultaneously perform nitrous acid degradation and complete enzymatic digestion by using heparinases I, II, and III on samples, which greatly improves analysis efficiency, and meanwhile, obtains more complete structural information of basic building blocks, especially the epimeric information of IdoA and GlcA in the basic building blocks.

2. At present, the sequencing of LMWHs still has technical limitations. For a group of complex heparin oligosaccharide mixtures, at present, there is no efficient and convenient method for sequencing them one by one. The present disclosure provides rapid and high-throughput sequencing method of a group of LMWH oligosaccharide mixtures with similar structures and compositions, so that sequences and contents of mixed heparin oligosaccharides can be quickly obtained.

Although the present disclosure has been described in detail with reference to the foregoing embodiments, those of ordinary skill in the art should understand that they can still modify the technical solutions described in the foregoing embodiments, or equivalently replace some of the technical features; and these modifications or replacements does not cause the essence of the corresponding technical solutions to deviate from the spirit and scope of the technical solutions of the embodiments of the present disclosure.

What is claimed is:

1. A sequencing method of low molecular weight heparin (LMWH) oligosaccharides, comprising:
a sample preparation step: isolating or preparing a group of LMWH oligosaccharide mixture samples according to experimental requirements;
a sample treatment step: performing complete enzymatic digestion and nitrous acid degradation on the LMWH oligosaccharide mixture samples to obtain an enzymatically digested eight-common-heparin-disaccharide array, a 3-O-sulfate group array, a 1,6-anhydro structure array, a nitrous acid degradation array, respectively;
a data processing step: calculating IdoA/GlcA of different disaccharides according to the eight-common-heparin-disaccharide array and the nitrous acid degradation array to obtain a disaccharide isomeric unit array;
a sequence database building step: building a sequence database according to a degree of polymerization of the oligosaccharide mixture, the disaccharide isomeric unit array, the 3-O-sulfate group array, and the 1,6-anhydro structure array; and
a specific result output step: screening the sequence database according to input qualification information and then outputting a specific result file.

2. The sequencing method of LMWH oligosaccharides according to claim 1, wherein the sample treatment step comprises:
an enzymatic digestion treatment step: performing the complete enzymatic digestion on the LMWH oligosaccharide mixture samples with a mixture of heparinases I, II, and III to obtain the enzymatically digested eight-common-heparin-disaccharide array, the 3-O-sulfate group array, and the 1,6-anhydro structure array; and
a nitrous acid degradation treatment step: degrading the LMWH oligosaccharide mixture samples with nitrous acid to obtain the nitrous acid degradation array.

3. The sequencing method of LMWH oligosaccharides according to claim 1, wherein the sequence database building step comprises:
a 1,6-anhydro structure-excluding theoretical sequence database building step: building a 1,6-anhydro structure-excluding theoretical sequence database according to the 3-O-sulfate group array and the disaccharide isomeric unit array;
a 1,6-anhydro structure-including theoretical sequence database building step: building a 1,6-anhydro structure-including theoretical sequence database according to the 3-O-sulfate array, the 1,6-anhydro structure array, and the disaccharide isomeric unit array; and
a combination step: correlating and combining the 1,6-anhydro structure-excluding theoretical sequence database and the 1,6-anhydro structure-including theoretical sequence database to obtain the sequence database.

4. The sequencing method of LMWH oligosaccharides according to claim 1, wherein the result file comprises: 1,6-anhydro structure-including sequences—generation time, 1,6-anhydro structure-excluding sequences—generation time, total sequences—sorted in decreasing order based on the content—generation time, selected sequences—at least one of generation time and a log file that comprises at least one of the total number of calculated sequences, the type and value of the disaccharide isomeric unit array, the total record number of total sequences, the total record number of 1,6-anhydro structure-excluding sequences, and the total record number of 1,6-anhydro structure-including sequences.

5. The sequencing method of LMWH oligosaccharides according to claim 2, wherein the 1,6-anhydro structure-excluding theoretical sequence database building step comprises: if all elements in the 3-O-sulfate group array are 0, setting the number of selected elements as dp/2, selecting dp/2 elements from the disaccharide isomeric unit array, arranging all the selected elements in a row in order, and calculating and outputting string concatenation of various elements and a product of values of various elements; and if all elements in the 3-O-sulfate group array are not 0, setting the number of selected elements as (dp−4)/2, selecting at least one element from the 3-O-sulfate group array, selecting (dp−4)/2 elements from the disaccharide isomeric unit array, arranging the remaining selected elements in a row in order, and calculating and outputting string concatenation of various elements and a product of values of various elements, wherein dp is the degree of polymerization of the oligosaccharide mixture.

6. The sequencing method of LMWH oligosaccharides according to claim 2, wherein the 1,6-anhydro structure-including theoretical sequence database building step comprises:
if an IdoA2S-GlcNS6S-IdoA2S-ManNS-1,6-anhydro group in the 1,6-anhydro structure array is greater than 0 and all elements in the 3-O-sulfate group array are 0, setting the number of selected elements as (dp−4)/2, selecting one element (IdoA2S-GlcNS6S-IdoA2S-ManNS-1,6-anhydro) from the 1,6-anhydro structure array, selecting (dp−4)/2 elements from the disaccharide isomeric unit array, setting the element (IdoA2S-GlcNS6S-IdoA2S-ManNS-1,6-anhydro) as the rightmost element of a string, arranging the (dp−4)/2 elements that are selected from the disaccharide isomeric unit array in a row in order on the left side of the element, and calculating and outputting string concatenation of various elements and a product of values of various elements;
if an IdoA2S-GlcNS6S-IdoA2S-ManNS-1,6-anhydro group in the 1,6-anhydro structure array is greater than 0 and all elements in the 3-O-sulfate group array are not 0, setting the number of selected elements as (dp−4-4)/2, selecting at least one element from the 3-O-sulfate group array, selecting one element (IdoA2S-GlcNS6S-IdoA2S-ManNS-1,6-anhydro) from the 1,6-anhydro structure array, selecting (dp−4−4)/2 elements from the disaccharide isomeric unit array, setting IdoA2S-GlcNS6S-IdoA2S-ManNS-1,6-anhydro as the rightmost element of a string, arranging the remaining selected elements in a row in order, and calculating and outputting string concatenation of various elements and a product of values of various elements;
if a GlcA-GlcNS/ManNS-1,6-anhydro group or an IdoA2S-GlcNS-1,6-anhydro group in the 1,6-anhydro structure array is greater than 0 and all elements in the 3-O-sulfate group array are 0, setting the number of selected elements as (dp−2)/2, selecting one element (GlcA-GlcNS/ManNS-1,6-anhydro or IdoA2S-GlcNS-1,6-anhydro) from the 1,6-anhydro structure array, selecting (dp-2)/2 elements from the disaccharide isomeric unit array, setting GlcA-GlcNS/ManNS-1,6-anhydro or IdoA2S-GlcNS-1,6-anhydro as the rightmost element of a string, arranging the (dp−2)/2 elements that are selected from the disaccharide isomeric unit array in a row in order on the left side of the element, and calculating and outputting string concatenation of various elements and a product of values of various elements; and
if a GlcA-GlcNS/ManNS-1,6-anhydro group or an IdoA2S-GlcNS-1,6-anhydro group in the 1,6-anhydro structure array is greater than 0 and all elements in the 3-O-sulfate array are not 0, setting the number of selected elements as (dp−4−2)/2, selecting one element (IdoA-GlcNAc6S-GlcA-GlcNS3S6S or IdoA-GlcNS6S-GlcA-GlcNS3S6S or IdoA-GlcNAc6S-GlcA-GlcNS3S or IdoA2S-GlcNAc6S-GlcA-GlcNS3S6S or IdoA2S-GlcNS6S-GlcA-GlcNS3S) from the 3-O-sulfate group array, selecting one element (GlcA-GlcNS/ManNS-1,6-anhydro or IdoA2S-GlcNS-1,6-anhydro) from the 1,6-anhydro structure array, selecting (dp−4−2)/2 elements from the disaccharide isomeric unit array, setting GlcA-GlcNS/ManNS-1,6-anhydro or IdoA2S-GlcNS-1,6-anhydro as the rightmost element of a string, arranging the remaining selected elements in a row in order, and calculating and outputting string concatenation of various elements and a product of values of various elements.

7. A sequencing system of LMWH oligosaccharides, comprising:
a sample preparation unit, configured to isolate or prepare a group of LMWH oligosaccharide mixture samples according to experimental requirements;
a sample treatment unit, configured to perform complete enzymatic digestion and nitrous acid degradation on the LMWH oligosaccharide mixture samples to obtain an enzymatically digested eight-common-heparin-disaccharide array, a 3-O-sulfate group array, a 1,6-anhydro structure array, and a nitrous acid degradation array, respectively;
a data processing unit, configured to calculate IdoA/GlcA of different disaccharides according to the eight-common-heparin-disaccharide array and the nitrous acid degradation array to obtain a disaccharide isomeric unit array;
a sequence database building unit, configured to build a sequence database according a degree of polymerization of the oligosaccharide mixture, the disaccharide isomeric unit array, the 3-O-sulfate group array, and the 1,6-anhydro structure array; and
a specific result output unit, configured to screen the sequence database according to input qualification information, and then output a specific result file.

8. A sequencing kit for sequencing mixed oligosaccharides of LMWH drugs, comprising H₂SO₄, Ba(NO₂)₂, NaNO₂, Na₂CO₃, acetic acid, ammonium hydroxide, NaBH₄, heparinase I, heparinase II, heparinase III, and an enzymatic digestion buffer.

9. The sequencing kit according to claim 8, specifically comprising:
1 mL of H₂SO₄ with a concentration of 0.5 moL; 1 mL of Ba(NO₂)₂ with a concentration of 0.5 moL; 1 mL of NaNO₂ with a concentration of 5.5 moL; 1 mL of Na₂CO₃ with a concentration of 1.0 moL; 1 mL of acetic acid with a concentration of 0.1 moL; 1 mL of ammonium hydroxide with a concentration of 0.1 moL; 1 g of NaBH₄; 20 mIU 50 μL*10 of heparinase I; 20 mIU 50 μL*10 of heparinase II; 20 mIU 50 μL*10 of heparinase III; and 1 mL of enzymatic digestion buffer.

10. The sequencing kit according to claim 8, wherein the sequencing kit is used to perform nitrous acid degradation on a LMWH drug to obtain a nitrous acid degradation product, and is used to perform complete enzymatic digestion on the LMWH drug to obtain a complete enzymatic digestion product.
